(19) United States
(12) Patent Application Publication
(10) Pub. No.: US 2009/0197818 A1
(43) Pub. Date: Aug. 6, 2009

(54) Use Of Ribose for Recovery From Anaesthesia

(76) Inventors: John A. St. Cyr, Coon Rapids, MN (US); David J. Perkowski, San Clemente, CA (US)

Correspondence Address: Kathleen R. Terry;Bioenergy, Inc., 13840 Johnson Street NE, Ham Lake, MN, 55304, US

(21) Appl. No.: 10/585,961

(22) PCT Filed: Jan. 14, 2005
(86) PCT No.: PCT/US2005/001435

§ 371 Date: Sep. 29, 2008

Related U.S. Application Data
(60) Provisional application No. 60/536,460, filed on Jan. 14, 2004.

(51) Int. Cl. A61K 31/7004; C07H 3/02

(52) U.S. Cl. 514/23; 536/1.11

(57) ABSTRACT

D-Ribose is administered before and after general anaesthesia to reduce the time to recover from the effects of general anaesthesia. Preferably, pyrogen-free D-Ribose is administered intravenously during general anaesthesia and the interval post-anaesthesia before oral administration can be resumed. D-Glucose may be co-administered to reduce the effect of hypoglycemia that may be seen with D-Ribose administration.

### CROSS-REFERENCE TO RELATED APPLICATIONS

This invention is related to and takes priority of Provisional Application Ser. No. 60/536,460, filed Jan. 14, 2004.

### BACKGROUND OF THE INVENTION

It is well known that the pentose sugar ribose is important in the energy cycle as a constituent of adenosine triphosphate (ATP) and nucleic acids. It is also well known that ribose is found only at low concentrations in the diet, and that further, the metabolic process by which the body produces ribose, the pentose phosphate pathway, is rate limited in many tissues.

Ribose is known to improve recovery of healthy dog hearts subjected to global ischemia at normal body temperatures, when administered for five days following removal of the cross clamp. These inventors have previously discovered (U.S. Pat. No. 6,159,942) that the administration of ribose enhances energy in subjects who have not been subjected to ischemic insult. In the case of human patients, by the time cardiac surgery is necessary, the condition of the heart and, possibly, the general state of health, are both impaired. Morbidity and mortality following myocardial ischemia which provides a dry working field can increase due to tissue damage. In addition, the patient is under anesthesia for a considerable period of time.

Most anaesthetic techniques act by inducing a reversible disturbance of the central nervous system (CNS). Spinal or epidural application of local anaesthetics produce a localized inhibition of impulse transmission at spinal cord level leading to central nervous blockade where the essential features are segmental loss of sensory and motor function. General anaesthetics administered intravenously act through binding to specific receptors such as opioid or GABA (γ-aminobutyric acid) receptors; however, the mechanisms of action for inhaled anaesthetics are less well described. Regardless of whether the anaesthetic is local or general, depression of CNS function is intended as part of the anesthesia. All bodily processes are slowed down by the CNS depression. In addition, it is usually necessary during extensive surgery to intubate the patient for respiratory support due to paralysis caused by administration of a curare-type drug. In spite of the respiratory support, pulmonary function is less than optimum. The reduced muscle tone of the diaphragm and intercostal muscles leads to atelectasis, with resulting hypoxemia. The reduced or absent muscle tonus of the skeletal muscles may also lead to reduced circulation and localized hypoxia. Likewise, other organ functions such as the kidney and liver function are somewhat suppressed, leading to accumulation of toxic metabolites. In the worse case scenario, brain dysfunction may be irreversible and manifested by subtle loss of cognitive ability, stroke or irreversible coma or cerebral death.

Upon recovery from anesthesia, the patient usually experiences mental and physical compromise for a period of time. For the first month post anesthesia, it is common for the patient to require more sleep, be less alert when awake and have diminished physical strength. Recurring pain from surgery may necessitate the administration of powerful analgesics which can worsen the already compromised mental and physical state.

It would be beneficial to patients undergoing surgery or any intervention requiring general anesthesia to have less impairment of function following anesthesia and a quicker recovery to normal alertness, ambulatory function and strength.

### SUMMARY OF THE INVENTION

D-Ribose is administered as a single agent or more preferably in combination with D-Glucose to a patient scheduled for a procedure requiring general anaesthesia. The agent or agents are administered before and after the general anaesthesia. Preferably, the agent or agents are administered before, during and after the general anaesthesia. Most preferably, the agent or agents are administered for one to seven days before surgery, during surgery and for one to seven days following surgery. The agent or agents are administered orally to a patient able to ingest a solution and intravenously during periods when intravenous fluids are administered.

A method of preparation of substantially pure, pyrogen-free ribose suitable for intravenous administration is disclosed. The intravenous dosage given of each agent or agents is from 30 to 300 mg/kg/hour, delivered from a solution of from 5 to 30% w/v of pyrogen-free D-Ribose in water. When D-Glucose is to be co-administered, it may be delivered from a solution of from 5 to 30% w/v of D-Glucose in water. The agent or agents to be administered are tapped into an intravenous line and the flow set to delivered from 30 to 300 mg/kg/hour agent or agents. Most preferably, pyrogen-free D-Ribose is administered with D-Glucose, each being delivered intravenously at a rate of 100 mg/kg/hour. When the agent or agents are administered orally, from one to 20 grams of D-Ribose is mixed in 200 ml of water and ingested one to four times per day. Most preferably, five grams of D-Ribose and five grams of D-Glucose are dissolved in water and ingested four times per day.

Patients in the intensive care unit (ICU) are administered pyrogen-free D-Ribose as a single agent or more preferably in combination with D-Glucose. The agent or agents are administered intravenously during the stay in the ICU. The intravenous dosage to be given of each agent or agents is from 30 to 300 mg/kg/hour, delivered from a solution of from 5 to 30% w/v of pyrogen-free D-Ribose in water. When D-Glucose is to be co-administered, it may be delivered from a solution of from 5 to 30% w/v of D-Glucose in water. The agent or agents to be administered are tapped into an intravenous line and the flow set to delivered from 30 to 300 mg/kg/hour agent or agents. Most preferably, pyrogen-free D-Ribose is administered with D-Glucose, each being delivered at a rate of 100 mg/kg/hour. When patients are released from the ICU, it is beneficial to continue the administration of the agent or agents. Intravenous administration will be continued while an IV line is in place. When the agent or agents are administered orally, from one to 20 grams of D-Ribose is mixed in 200 ml of water and ingested one to four times per day. Most preferably, five grams of D-Ribose and five grams of D-Glucose are dissolved in water and ingested four times per day.

### DETAILED DESCRIPTION OF THE INVENTION

The following examples are given to show how the invention has been or is to be practiced. Those skills in the art can readily make insubstantial changes in the methods and compositions of this invention without departing from its spirit and scope. In particular, it will be noted that in most of the examples, it is suggested that D-Glucose be given along with D-Ribose. It should be noted that the administration of D-Glucose is advised not as a therapy, but to avoid the hypoglycemia that can occur when D-Ribose is given. If it has been determined that a particular subject does not show hypoglycemia on D-Ribose administration, the D-Glucose may be eliminated. It is suggested that the agent be given one to seven days before and one to seven days after anaesthetic is delivered. Many subjects may have self-administered ribose for a longer period. Therefore the method is not limited to the minimal times given, but includes long-term ribose administration both before and after the anaesthetic procedure. Most importantly, the term ribose must be taken to include D-Ribose and other related compounds that are readily converted to ribose in vivo or which spare endogenous ribose. These compounds include ribitol, ribulose, 5-phosphoribose, xylitol, xylulose and sedoheptulose.

### EXAMPLE 1

### Preparation of Substantially Pure, Pyrogen-Free Ribose

Products produced by fermentation generally have some residue of pyrogens, that is, substances that can induce fever when administered intravenously. Among the most frequent pyrogenic contaminants are bacterial endotoxins. Therefore, endotoxin analysis is used to determine whether a substance is or is not essentially free of pyrogens. Additionally, congeners, that is, undesirable side products produced during fermentation and heavy metals may be carried through and present in the fermentation product.

D-Ribose prepared by fermentation and purified is approximately 97% pure and may contain varying levels of endotoxin. While this product is safe for oral ingestion and may be termed “food grade” it is not “pharma grade,” suitable for intravenous administration. D-Ribose may be purified to pharma grade and rendered pyrogen-free. Briefly, all equipment is scrupulously cleaned with a final rinse of pyrogen-free water, which may be double distilled or prepared by reverse osmosis. All solutions and reagents are made up with pyrogen-free water.

A solution of about 30% to 40% ribose in water is prepared. Activated charcoal is added and the suspension mixed at least 30 minutes, while maintaining the temperature at 50-60° C. The charcoal is removed by filtration. The filtered solution should be clear and almost colorless.

Ethanol is added to induce crystallization and the crystals allowed to grow for one or two days. For convenient handling, the crystals are ground and transferred to drums, bags or other containers. Each container is preferably supplied with a bag of desiccant. The final product is essentially pure and free of pyrogens, heavy metals and congeners.

### EXAMPLE 2

### Enhancement of Recovery of Myocardial Function Following Global Cardiac Ischemia

Global myocardial ischemia during cardiac surgery rapidly depletes myocardium high energy phosphate stores. ATP is rapidly catabolized to purine bases, which readily permeate the cell membrane and are not available to the most efficient pathway, the salvage pathway, for the resynthesis of ATP when the circulation is restored. Thus, restitution of depleted myocyte ATP following cardiac surgery relies primarily on de novo synthesis of adenine nucleotides through the oxidative pentose phosphate pathway. Zimmer (Zimmer et al., J. Mol. Cell. Cardiol. 16(9) 863-866, 1984) has provided a complete review of the oxidative pentose phosphate pathway. In summary, the availability of 5-phosphoribosyl-1-pyrophosphate (PRPP) determines the rate of synthesis of the adenine nucleotides. PRPP production, in turn, depends on the activity of glucose-6-phosphate dehydrogenase, the first and rate limiting enzyme in the pentose phosphate pathway. The administration of D-Ribose, a pentose sugar, bypasses the rate limiting step and thereby enhances the resynthesis of ATP.

Foker (U.S. Pat. No. 4,719,201) found that healthy dog hearts require up to nine days to establish normal baseline ATP levels following a 20 minute, normothermic period of global myocardial ischemia. Administration of D-Ribose immediately at reperfusion and continuing for at least four days enhanced ATP recovery. A protocol was devised to test whether human subjects undergoing either valve surgery plus coronary artery bypass graft (CABG) or CABG alone with decreased heart function would benefit from the administration of ribose following heart surgery as did the healthy dogs of the Foker study.

After FDA and institutional review board approval, informed consent was obtained from 49 patients for enrolment in a prospective single center, double-blind, placebo-controlled clinical trial, designed to evaluate the efficacy of intravenous D-Ribose for the treatment of myocardial dysfunction resulting from globally induced ischemia during cardiac surgical procedures.

Inclusion criteria were:
Males or females aged 18 or older
Patients with documented coronary artery disease undergoing CABG with an ejection fraction (EF) of ≦35% based on echocardiography, radionuclide imaging or cardiac catheterization done within eight weeks of surgery. (If more than one method was used to evaluate EF during this period, the mean values of the various methods were ≦35%).
Patients undergoing single or double valve replacement with documented coronary artery disease also undergoing CABG; or patients undergoing single or double valve replacement without CABG
Serum creatinine of <2.35 mg/dl
For females of childbearing potential, a negative pregnancy test.
Signed consent forms.

The test article, placebo or ribose, was dispensed according to computer-generated randomization schedule either for patients undergoing CABG only or for patients undergoing heart valve surgery +/−CABG. All patients received a high dose narcotic anaesthesia technique consisting of either fentanyl (50-100 μg/kg) or sufentanil (10-20 μg/kg) and midazolam. No restriction was placed on the type of anaesthetic agents administered. The anaesthesiologists and surgeons responsible for the care of the patents made the clinical decision to use inotropic support, intra-aortic balloon pump support or post bypass circulatory support based on their knowledge of patients requirements and accepted medical practice and without regard to test article status. The test article infusion was started intravenously at the time of aortic cross clamping and continued until the pulmonary artery catheters introducer was removed or for five days (120) hours whichever occurred first. The surgeons responsible for the clinical care of the patients removed the pulmonary artery catheter cordis without regard to test article stats.

Hemodynamic measurements consisting of heart rate, blood pressure, pulmonary artery pressures, pulmonary capillary wedge pressure (PCWP), central venous pressure (CVP) and thermodilution cardiac index (CI) were obtained at the following time intervals: immediately prior to induction of anaesthesia, post induction of anaesthesia prior to sternotomy, post sternotomy prior to initiation of cardiopulmonary bypass, upon successful termination of cardiopulmonary bypass prior to sternal closure and prior to reversal of heparinization with protamine, post closure of the sternum, upon arrival in the intensive care unit and at one or two hour intervals until the pulmonary artery a catheter was removed.

Transesophageal echocardiography data (H.P. Sonos OR, 5.0 MHz, Andover, Mass.) was collected at the following time intervals: post induction of anaesthesia prior to sternotomy, and immediately post closure of the sternum. Transthoracic echocardiography (H.P. Sonos 1500. 2.5 MHz, Andover, Mass.) measurements were made on day three and day seven of the study period. For both the transesophageal and transthoracic echocardiograms, the following long axis and short axis mid-papillary area changes were measured in triplicate by acoustic quantification techniques: end diastolic area (EDA), end systolic area (ESA), fractional area change (FAC), +dA/dt and −dA/dt. All area change data were also analyzed by manual off line analysis. EF was also determined off line using a long axis view. In addition, regional wall motion was quantified as the following: normal=1, hypokinetic=2, akinetic=3 and dyskinetic=4. The wall motion index score (WMIS) and percentage normal myocardium were calculated by reading a maximum of sixteen segments. Echocardiography data for evaluating wall motion and area change was analyzed only if greater than 75% of the endocardial border could be visualized through a complete cardiac cycle. Off line analysis was performed on an Image View echocardiography workstation (Nova Microsonics, Allendale, N.J.). Transmitral Doppler flow velocity measurements made at the level of the mitral valve leaflets included early diastolic filling (E), the atrial filling component (A) and the E/A ratio. Valvular insufficiency was evaluated and quantified as none, trace, mild, moderate, or severe. An interpreter blinded to both treatment and outcome analyzed all echocardiography data.

All concomitant medications given within 24 hours of the test article and up through Day 7 were recorded including indication, time started, time completed and total dose(s). Input (NG, oral and intravenous fluids) and outputs (urine and other fluids) were measured and recorded through Day 7 as available per hospital routine.

Clinical outcome parameters included the following: number of attempts to wean from CPB, time to extubation, time to discharge from the ICU, time to hospital discharge, number and duration of inotropic drugs, use and duration of intraaortic balloon pump support, and survival to to 30 days postoperatively.

Blood glucose levels were determined hourly, after initiation of the study drug infusion, by dextrastix (Accu-Chk III, Boehringer Mannheim Corp. Indianapolis Ind.) using blood from an intraarterial catheter. If the blood glucose level remained stable for 12 hours, then subsequent blood glucose levels were measured every 4 to 6 hours until the study drug infusion was stopped. Other clinical laboratory measurements including complete CBC with differential, platelet count, electrolytes=, liver function studies, serum osmolarity, and urinalysis were completed the morning following surgery. Abnormal laboratory tests were repeated as clinically indicated until normal or determined not to be clinically significant.

All data were entered into a Microsoft Excel Spreadsheet (v4.0, Microsoft Corp., Redmond, Wash.). Before unblinding, 100% of the echocardiography data, 20% of the hemodynamic data and 5% of all other data were audited. The entry error rate was less than 0.001%. A detailed statistical analysis plan for evaluation of the demographic, safety, and efficacy data was developed before unblinding of the study. All statistics were computed on JMP software (v3.1 for Windows, SAS Institute Inc., Cary, N.C.). The plan excluded those patients deemed not possible to evaluate because of protocol violations including interruption of test article administration for greater than a four-hour period (one subject), technically limited echocardiographic studies, and interoperative surgical difficulty not related to pharmacological treatment (two subjects). Covariates included age, aortic cross clamp time, baseline EF, and baseline WMIS. Statistical tests included Chi square, t-test, univariate ANOVA for repeated measures, and ANCOVA. For all statistical tests p<0.05 (two-tailed) was considered to represent statistical significance.

After the inclusion of 49 patients, the enrollment of additional patients was suspended because of an institutional decision to extubate all cardiac surgery patients within six hours postoperatively and discharge the patients from the ICU within 24 hours, if clinically stable. This decision required an alteration of anaesthetic technique and postoperative management. As a result of early this termination of the study, we excluded from analysis nine enrolled patients, including those patients with isolated mitral insufficiency (n=3), isolated mitral stenosis (n=3), combined aortic and mitral valve disease (n=3).

The demographic and baseline measurements of cardiac function for those patients for whom both baseline and day 7 EF could be determined by echocardiography and who had aortic stenosis or coronary artery disease (n=27) was examined. The ribose treated patients were older (66.5 yr. vs. 56.4 yr, p=0.026) and tended to have a lower baseline EF than the placebo treated patients. However, the baseline difference in EF did not achieve statistical significance. Other significant baseline differences were not found for these patients.

The mean baseline EF for placebo treated patients declined from 55% to 38% at Day 7 (p=0.0025). The mean baseline and Day 7 EF for the ribose treated patients was unchanged (44% vs. 41%, p=0.49). The split-plot time effects of treatment group on EF as calculated from a univariate ANOVA model for repeated measures with random effect was statistically different (prob>F, p=0.04). EF was maintained in the ribose treated patients whereas in placebo treated patients, EF declined. The hypothesis tests provided by JMP agree with the hypotheses tests of SAS-PROC GLM (types III and IV).

Five patients (28%) in the ribose treated group developed hypoglycemia (fingerstick glucose<70 mg/dl)) a known side effect of this pentose sugar. No placebo treated patients developed hypoglycemia. The mean glucose level in those patients developing hypoglycemia was 58 mg/dl. The lowest glucose level was 31 mg/dl. Three subjects were treated with a bolus injection of D50W; one subject was treated with oral apple juice; one subject did not require treatment. The study drug infusion was stopped in two subjects because of hypoglycemia. None of these patients developed neurological or other clinical symptoms associated with hypoglycemia. There were no statistical differences in the other clinical laboratory measurements. It is important to note that analysis including those subjects who had protocol violations did not alter any statistical outcome.

This study demonstrates the potential benefit of D-Ribose infusion at 100 mg/kg/hr for the preservation of postoperative EF in patients who have undergone aortic cross clamping. The EF decreased from baseline in the placebo treated patients whereas in the ribose treated patients, EF was maintained. It may be noted that although randomization was performed using standard methods, in this population group, the patients receiving ribose had a lower EF. Nonetheless, the EF was maintained while the higher EF of the placebo controls decreased.

### EXAMPLE 3

### Preconditioning with D-Ribose before Cross Clamping

Example 2 demonstrates that administration of D-Ribose intravenously during and after cross clamping of the aorta maintains and improves EF compared to administration of D-glucose. A single-center, randomized, double-blinded placebo-controlled clinical trial was designed to determine if preoperative oral administration of D-Ribose, following by peri-operative and operative intravenous infusion of D-Ribose could improve the ejection fraction and other functional parameters of hearts that are cross-clamped for various cardiac surgical procedures.

Thirty (30) patients meeting the inclusion and exclusion criteria and who have signed informed consent forms will be randomized to receive oral D-Ribose (15) or D-Glucose (placebo) (15) for seven or 14 days prior to their surgical procedure and intravenous 5% D5NS (5% D-Glucose in normal saline, 0.5 mL/kg/hour) or 10% pyrogen-free D-Ribose in 5% D5W at a dose of 100 mg/kg/hour for five (5) days through a pulmonary artery cordis) beginning at the time of aortic cross clamping. (In the event that the pulmonary artery catheter is removed prior to the end of the five day infusion, the remaining test article will be administered through a peripheral intravenous (IV) line.) Patients randomized to the D-Ribose group will receive oral and IV test supplement and those randomized to placebo will receive oral and IV D-Glucose. Patients will be evaluated baseline ×2, (once prior to beginning oral test supplement and again within three days prior to surgical procedure), during and after surgery, and at days 1, 5 and 7. The discharge date will be noted.

Inclusion criteria include:
Ages 18 or older, males and females
Patients with documented aortic valve disease, undergoing AVR, with EF of ≦35% based on echocardiography, radionuclide imaging or cardiac catheterization done within four weeks prior to surgery. If more than one method was used to evaluate EF during this period, the mean values will be ≦35%.
Serum creatinine <2.5 mg/dl.
For females of child bearing potential, a negative pregnancy test within two weeks prior to surgery.
Signed consent form which has been approved by the Institutional Review Board at the investigational site.

Exclusion criteria include:
Clinically significant chronic obstructive lung disease requiring bronchodilators.
Cardiogenic shock requiring inotropic support preoperatively.
Clinically significant liver disease.
Esophageal pathology that precludes transesophageal echocardiography.
Pregnant females.

A randomization schedule will be generated and given to the institutional pharmacy for preparation of the test article and placebo. At the time of randomization, patients will be sequentially assigned a number from the randomization schedule. In addition to the assigned number, the patients will be identified by their initials in the pharmacy records only.

If an adverse reaction occurs and the investigator believes that the identity of the test article is necessary information for treatment decisions, an independent reviewer (physician) will be informed by the Pharmacy of the identity of the test article. The unblinding will be documented in the pharmacy's records and the patient's case report form. The reviewer will make the determinations of the relationship of the adverse reaction to the test article.

The study will proceed as follows:

Patients will be evaluated for eligibility within three days to first test article administration and evaluation will be updated within three days prior to surgery. Ejection fraction determination within the past four weeks will be reviewed. The type of test, date of the test and results will be entered into the case report. Informed consent and a limited medical history will be obtained to assess preoperative risk factors including prior open heart surgery, cerebrovascular disease, prior vascular surgery, history of angina, cigarette smoking and alcohol use. A medication history will be taken and all medications recorded in the case report. This medication history will be updated prior to surgery. A limited physical examination will be carried out and will include blood pressure, weight, and examination of the heart, lungs and extremities. Laboratory studies, including a complete blood count (CBC, Hgb, Hct, RBC, WBC with differential, platelet count), creatinine, BUN, blood sugar (Glucose), Na, K, Cl, CO2, AST, ALT, bilirubin, calcium, PO4, serum osmolarity and urinalysis), will be obtained some time during the three days prior to surgery. An electrocardiogram will be done within three days prior to surgery. A baseline transthoracic echocardiogram will be done within the 14 day period prior to first test article administration. Once patients have signed an informed consent form and satisfied the initial screening, they will be randomized to receive either D-Ribose or D-Glucose for 7 days prior to surgery.

Following the seven day oral administration of test article or placebo, patients will be admitted for surgery. Prior to anaesthesia, post induction of anaesthesia (prior to sternotomy) and post sternotomy prior to initiation of cardio-pulmonary bypass (CPB), hemodynamic measurements (CI, CVP, pulmonary wedge pressure, PA pressure, blood pressure) will be obtained. Transesophageal echocardiography will be performed post induction of anaesthesia (prior to sternotomy). The duration of aortic cross clamp time will be recorded in the case report forms.

The IV test article and placebo will be started at the time of aortic cross clamping. In order to avoid the hypoglycemic effects seen in some patients of Example 1, D-Glucose will be co-administered with D-Ribose. The infusion of 10% D-Ribose plus 5% D-Glucose or placebo equivalent will be given through the pulmonary artery catheter cordis at a rate that delivers 100 mg/kg/hour of D-Ribose or placebo equivalent. The IV test infusion will continue for five days.

Hemodynamic measurement will be repeated at the following time points:
Upon successful termination of CPB, prior to sternal closure.
Upon reversal of heparin with protamine.
Post closure of the sternum.
Upon arrival in the postoperative ICU.
At hourly intervals until the investigator concludes that the patient is hemodynamically stable.
At two hour intervals until the pulmonary artery catheter is removed.

Transesophageal echocardiography will also be done post closure of the sternum. Transthoracic echocardiography will be performed on postoperative days 1, 5 and 7. M-mode, two-dimensional and Doppler echocardiography will be used to assess left ventricular (LV) systolic and diastolic myocardial function. The following measurements will be recorded for each assessment:

Measurements:
Standard M-mode measurements and calculations according to cardiology guidelines.
Left atrium two-dimensional anterior-posterior diameter, superior-inferior diameter and medial-lateral diameter.
Left ventricle volume, using Simpson's rule.
Right ventricle two-dimensional chamber sizes from both apical two and four chamber views.
Right atrium two-dimensional inferior-superior diameter and medial-lateral diameter.

The ventricular EF and stroke volume (SV) will be calculated:

LVSV=LV end-diastolic volume minus LV end-systolic volume

LVEF=LVSV/LV end-diastolic volume.

Diastolic function will be assessed using the flow velocity profile over the mitral valve and pulmonary venous flow. The use of contrast medium may be necessary to improve signal quality and reproducibility. The parameters will be calculated as follows:
Mitral inflow: Peak velocities during early (E_{V}) and late (A_{V} wave) diastolic, velocity time integral during early (E_{VTI}) and late (A_{VTI}) diastole, duration of early (E_{T}) and late A_{T}) diastole.
Pulmonary venous flow: Peak systolic (S_{V}) and diastolic (D_{V}) flow velocities, velocity time integral during systole (S_{VTI}) and diastole (D_{VTI}) in the left atrium.
E/A ratio=E_{V}/A_{V}
E/A_{VTI}=E_{VTI}/A_{VTI}
S/D_{V}=S_{V}/D_{V}
S/D_{VTI}=S_{VTI}/D_{VTI}

Pulmonary artery pressure can be assessed with echocardiography if tricuspid and pulmonary insufficiency are present and using an assumed right atrial pressure of 10 mm Hg.

All concomitant medications given post IV test article administration in the operating room, including through day 5 of IV test article administration will be recorded in the case report form including indication, time started, time completed, and doses(s). If an intraortic balloon pump (IABP) is required, the time(s) of its use will be recorded until discharge from the ICU. Input NG, oral and intravenous fluids) and output (urine and other fluids) will be measured and recorded until discharge from the ICU. Significant intervention such as cardioversion, atrial pacing, defibrillation or reintubation will be recorded in the case report forms.

Electrocardiograph monitoring will be continuous in the operating room and ICU. Episodes of ventricular tachycardia, ventricular fibrillation and atrial arrhythmias requiring cardioversion or rapid pacing will be recorded in the case report form including duration of the event. A 12 lead EKG will be obtained before discharge. Blood glucose levels will be determined hourly, after IV infusion is initiated, by dextrastix using blood drawn from the intraarterial catheter until stable and then every 4 to 6 hours thereafter. Laboratory studies as outlined above will be performed the morning following surgery. Abnormal lab tests will be repeated as clinically indicated until normal or determined not to be clinically significant. Serum osmolarity will be measured at least every other day during the period of IV infusion. A physical exam will be repeated before discharge from the ICU.

The following endpoints will be considered indications of efficacy: time to extubation, time to discharge from the ICU, time to hospital discharge, inotropic support (drug(s) and duration of inotropic drug(s) and/or duration of IABP); survival or death up to 30 days postoperatively; cardiac indices; PA wedge pressures; transesophageal and transthoracic echocardiographic changes in contractility and wall motion abnormalities.

It is expected that as for Example 2, the patients receiving D-Ribose will have better myocardial function and may show shorter duration on inotropic drugs and/or IABP, and an earlier discharge from the ICU and hospital. Furthermore, the results seen in Example 1 will be enhanced by the oral preloading of the patient with D-Ribose.

### EXAMPLE 4

### Presurgical Loading with Ribose

Patients with stable coronary artery disease or patients presenting with acute myocardial infarction may undergo revascularization using an “off” cardiopulmonary bypass procedure (“OCBP”). This procedure avoids the deleterious effects of cross-clamping in patients who can be selected for the procedure. The selection criteria include: accessibility of the grafts; number of the grafts; condition of the patient. When the area to be revascularized is at the back of the heart, the heart will need to be handled and rotated, which would interfere with the beating of the heart. If several grafts are needed, the time of operation is prolonged and the patient's heart will need to support circulation for a long time. Finally, a poorly functioning heart will need the assistance of the pump to support circulation. Nevertheless, in properly selected patients, the benefits of avoiding cross-clamping are substantial.

Forty-four adult patients were enrolled in a trial. At least one-third of the patents had sustained an acute myocardial infarction prior to presentation and pre-operative ejection fractions ranged from 30-72%. All patients were selected for OCBP revascularization with 20 patients consuming no pre-operative ribose and 24 patients given oral D-ribose preoperatively, cardiac indices were measured at baseline and post-operatively. All cardiac medications and risk assessment scores (STC criteria). The ribose treated patients demonstrated a 49% greater increase in cardiac indices compared to controls (p<0.028).

### EXAMPLE 5

### Recovery from Anaesthesia

During deep anaesthesia, all bodily functions are depressed. After any prolonged general anaesthesia, that is, anaesthesia where the human patient is unconscious for at least three hours, recovery to full energetic state may require a full month or more. For purposes of describing this invention, by “recovery” is meant the ability of a patient subjected to general anaesthesia to resume normal alertness, ambulatory function and eating. If the patient experiences pain from a surgical procedure, an important aspect of recovery is relief from pain. Hendricks et al (Resuscitation 1984 November: 12(3):213-21, the teachings of which are hereby incorporated by reference) found that rats anesthetized with halothane for 30 minutes showed reduced spontaneous activity and neurological deficit during the first week after anaesthesia. The authors concluded that halothane and nitrous oxide have prolonged effects on locomotor behavior beyond the immediate post- recovery period. Similar effects are frequently observed in human patients after surgery. Patients find that they need more sleep, get fatigued easily throughout a day and are not alert enough to drive an automobile for several weeks. In addition, postoperative pain may require prolonged use of analgesic drugs, which may further inhibit physical activity, as patients tend to be more sedentary to minimize pain. As can be seen in Example 4, not all the effects shown in cardiac surgery wherein the heart is cross-clamped, with resultant decrease in heart function due to ischemia may be due to the ischemia alone. As noted, patients not subjected to ischemia and therefore assumed to have more normal heart function, also benefited from ribose administration as shown by better cardiac outcome. Other aspects of recovery from anaesthesia were not recorded in that trial. Trials were performed to determine whether the better function beyond cardiac parameters due to ribose administration can be shown in other cases of general anaesthesia.

A. Anecdotal results from Non-Cardiac Surgery with General Anaesthesia

Anecdotal reports have indicated that the administration of D-Ribose hastens recovery to a full energetic state and further, that the degree and duration of pain episodes seem to be lessened. For example, a 69 year-old woman underwent two hip replacement operations, five months apart. With the second operation, she began taking oral ribose immediately after her recovery from anaesthesia. Her recovery to a feeling of alertness and energy was more rapid than after the first operation. Furthermore, her level of pain was less. Likewise, a 52 year-old man also underwent two knee replacement operations. With the second operation, he self-administered D-Ribose pre- and postoperatively. His recovery to a feeling of alertness and energy was more rapid than after the first operation. Bauer et al. (Z. Geb. Neonatal 2001 May-June, 205(3):80-85) studied the efficacy of oral glucose for treating procedural pain in neonates. They found that placing a solution of glucose on the tongue of the infant reduced the degree of pain experienced during venous blood sampling. The authors proposed that the orogustatory stimulation by the sweet taste caused an endorphin release. It is not known whether the result seen was due to the local effect or to a systemic effect of glucose.

### B. Cardiac Surgery, Sheep Study

A study on aortic valve replacement in sheep was carried out. Fourteen cross-bred (male and female) sheep (age range 25 to 68 weeks, body range 47 to 68 kg) were used in these studies. There were two postoperative deaths. The mean CPB time was two hours. (1 Heart Valve Disease Vol 9. No 6, November 2000, the teachings of which are incorporated by reference). The surgical protocol was as follows: Two days before surgery, each animal was given an intramuscular injection of antibiotic: ticarcillin disodium, 0.03 g/kg (SmithKline Beecham Pharmaceuticals, Philadelphia) and Gentocin 1 mg/kg (Fermenta Veterinary Products, Kansas City, Mo.). On the day of surgery, each animal was given an intramuscular injection of Gentocin 1 mg/kg and atropine sulfate (Medco, St. Joseph, Mo.), 5 ml of a 2% w/v solution in normal saline. A peripheral intravenous line was inserted. Sodium pentothal (2.5%, Abbott Laboratories, North Chicago, Ill.) and ticarcillin disodium (0.03 g/kg). General anaesthesia was maintained with isoflurane and supplemental oxygen with further doses of sodium pentothal administered as necessary. The animals were intubated and ventilatory support established. Succinylcholine was given before any incision was made.

The usual intrasurgical parameters were followed, among which were EEG, rectal and esophageal temperatures, serial arterial blood gas. The animal was placed on cardiopulmonary bypass using a Maxima® hollow fiber membrane oxygenator with venous reservoir pump and a BioMedicus 80 constrained vortex centrifugal pump. Cooling was initiated. When adequate cooling had occurred, an aortic cross clamp was applied across the distal ascending aorta and cold (4° C.) cardioplegia with 10 meq KCl (Plegisol, Abbott Laboratories) was administered proximal to the applied aortic cross clamp, ice slush was placed over and around the heart, which arrested immediately. The ascending aorta was completely transected transversely, proximal to the cross clamp. During the procedure, further doses of cardioplegia were administered at about 20 to 25 minute intervals directly into each coronary ostia. The aortic leaflets were excised and the annulus of the valve was sized for selection of the prosthetic valve. Prosthetic aortic valves (19 mm) were implanted in each animal, with interrupted, everting, abutting, mattress Ethibond suture being placed into the annulus of the aortic valve and thereafter placed into the skirt of the selected prosthetic valve. The transected aorta was reapproximated and sutured. The circulated blood was rewarmed to 42° C. and the heart defibrillated. Once the animal was off CPB and hemodynamically stable, the chest was closed. Ventilation was continued until the animal could breathe spontaneously. When the animal was judged to be alert, the endotracheal tube was removed. The mean time to extubation was about 3 to 4 hours after chest closure. Solid food was provided and the animals observed. The average animal remained quiet and inactive for an additional 2 hours and it was observed that food was not eaten until about 2½ to 3 hours after extubation.

In order to determine whether the administration of ribose could shorten the postsurgical recovery time, six cross-bred (male and female) sheep (age range 25 to 68 weeks, body range 46 to 65 kg) were administered pyrogen-free 5% D-Ribose in dextrose 5% water by intravenous infusion at a rate of 100 cc/hour from the time the pre-operative drip was inserted until it was withdrawn. In this series of surgical procedures, the mean time on CPB was slightly longer, from 2½ h to 3 hours. Nonetheless, the mean time to extubation was reduced to 1½ h to 2½ hours. The animals were monitored with cardiac output, blood pressure, and observation of myocardial relaxation state during and following cardioplegia, time to cardiac arrest with cardioplegia, the time interval between cardioplegic infusions, and the degree of vigorous contractility following defibrillation of the heart at the completion of surgery.

All animals tolerated the supplemental ribose with no metabolic or chemical abnormalities. The infusion of cardioplegia containing ribose resulting a somewhat faster cardiac arrest than in the animals not given ribose, the difference not being statistically significant. The heart was defibrillated easily. The heart was able increase function quickly and to be taken off bypass. Upon weaning from the ventilator, the animals were able to assume a consciousness state faster than the animals not given ribose, were then extubated, The animals were quiet and inactive for only about one hour, increased their activity by standing and even showing ambulaotary activity. Some began eating solid food within the next hour. Due to the increased activity and an interest in eating, it was assumed that the level of pain was less as was reported by the human subjects of Example 5A.

### C. Vascular Graft Placement

Adult sheep or canines will be used as an animal model for the effect of ribose on recovery of animals undergoing vascular grafts. The vascular grafts will vary, some being of artificial materials, such as Dacron, and some being of natural blood vessels taken from a donor animal. After passing the animal under general anaesthesia, as in Example 5B, a neck cutdown will be performed, isolating both the common carotid artery and jugular vein. An arterial catheter will be placed into the common carotid artery for blood pressure monitoring and subsequent blood sampling. A venous catheter will be placed into the jugular vein. Pyrogen-free D-Ribose or D-Glucose (each at 12.5 gm/l) will be administered intravenously at the commencement of the operation at a rate of 100 cc/hour. Both groins of the animal will be shaved, prepped and draped sterilely. Generous left and right groin cutdowns will be performed. Both femoral arteries (left and right) will be isolated and looped with umbilical tapes, both proximally and distally. Distal muscle biopsies will be obtained from both limbs of the animal./ these biopsies will be frozen immediately for adenine nucleotide analysis. The animal will receive acceptable systemic heparinization, as determined by ACT vaslues. A bolus 400 cc injection of pyrogen-free D-Ribose or D-Glucose (each 7 gm/l) will be performed. Vascular clamps will be applied both proximally and distally on each isolated femoral artery. A segment of native artery will be excised and an interposed segment of graft material will be tailored and sewn in place using a running suturing technique. Each anastomosis will incorporate two sutures, each running 180 degrees and tied to each other.

At the completion of each anastomosis, the vascular clamps will be removed in a specific order to make sure that any residual air has been evacuated. Another bolus 400 cc injection of pyrogen-free D-Ribose or D-Glucose (each 7 gm/l) will be given into the proximal femoral artery area. The same test substance will be used in the appropriate limb as determined at the time of the first bolus, prior to the anastomoses. Hemodynamic and fluoroscopic assessments will be made during the healing time to ascertain patency and integrity of the grafts.

The recovery of the animals will be monitored to determine whether the test animals can be extubated sooner, appear alert sooner and move voluntarily. Additional boliAnalgesics will be given for pain as indicated by the behavior of the animals.

### D. Non-Cardiac Surgery, Rat Study

In order to ascertain more definitively whether these results seen in sections B and C above are due to improvement in cardiac function or to improvement in the deficits due to general anaesthesia as indicated in section A above, the following study was designed. Littered-paired Wistar rats will be preconditioned with oral D-Ribose (250 mg/day, 10 animals) as a test drug or D-Glucose(250 mg/day, 10 animals) as a placebo for five days. Following the preconditioning, the rats will be anesthetized with halothane, intubated for artificial respiration and paralyzed with curare. Following general anaesthesia, the rats will be given either the test drug or placebo, intravenously (IV). A two-inch abdominal incision will be made and the viscera will be carefully manipulated to simulate an abdominal exploratory surgery. The incision will be closed and the animals will be held under anaesthesia for one additional hour. Following that hour, anaesthesia will be discontinued and the IV infusion will be halted. The animals will be placed individually in activity cages and their activity will be assessed daily for five days. Test drug or placebo will be added to the drinking water at a dosage of 5% wt/vol. The blinded results will be observed for: first movement (return to consciousness following the sham operation) and daily activity over the first day and next five days. Food and water intake and gastrointestinal function will be measured.

It is expected that the rats given D-Ribose before, during and after the sham operation will demonstrate earlier movement after anaesthesia and increased activity during the following five days, indicating that their recovery level is higher than that of the placebo controls and/or their experienced pain is lessened.

### EXAMPLE 5

### Use of D-Ribose to Minimize Dwell Time in the ICU

Patients are admitted to intensive care units (ICU) whenever their medical condition requires constant monitoring. Such gravely ill patients include those having experienced long-lasting surgery such as the cardiac surgical procedures of Examples 2, 3, 4 and 5B, or trauma from severe accidents and the like. Additionally, a common condition requiring ICU admittance is sepsis. Sepsis can be defined as a fulminant infection which has become disseminated throughout the body. Either the infective agent has established many foci of infection, is multiplying in the blood stream or has established one focus or a few foci of infection, from which toxins are perfused throughout the body. These toxins can cause multiorgan damage, often through inference with the integrity of cell membranes. If the infection is not controllable by antibiotic therapy and the bodily functions are not maintained by supportive therapy, the patient may go into shock, with plummeting blood pressure, multiorgan failure, progressing to death. The debilitated state of the tissues is reflected in low tissue ATP. Healthy humans, as shown in U.S. Pat. No. 6,159,942, can increase muscle ATP and recovery of ATP levels that are reduced during strenuous activity. A study will be designed to determine whether patients in the ICU with low ATP levels are able to benefit from ribose administration as an adjunct to the usual therapies for sepsis.

The compositions and methods of these examples are provided for instruction on the making and use of the present invention only and do not limit the scope of the appended claims. Those skilled in the art can readily make insubstantial changes to the compositions and methods of these examples without departing from the spirit and scope of the present invention.

1. A method of reducing recovery time of a mammal undergoing general anaesthesia comprising the administration of an effective amount of D-Ribose to said mammal.

2. The method of claim 1 wherein the effective amount of D-Ribose is administered orally before and after general anaesthesia.

3. The method of claim 2 wherein the effective amount of D-Ribose is from 2 to 10 grams and is administered two to four times daily.

4. The method of claim I wherein an effective amount of pyrogen-free D-Ribose is administered intravenously during and after general anaesthesia.

5. The method of claim 4 wherein the effective amount of D-Ribose is 20-300 mg/kg/hour.

6. A method of reducing recovery time of a mammal undergoing general anaesthesia wherein an effective amount of D-Ribose is administered orally to the mammal when the mammal is able to ingest the D-Ribose and an effective amount of pyrogen-free D-Ribose is administered intravenously to the mammal when the mammal is unconscious or otherwise unable to ingest the D-Ribose.

7. The method of claim 6 wherein the effective amount of D-Ribose to be administered orally is 2 to 10 gm and is administered two to four times daily and the effective amount of pyrogen-free D-Ribose to be administered intravenously is 20-300 mg/kg/hour.

8. A method for enhancing recovery from sepsis comprising of the administration of D-Ribose to the mammal suffering from sepsis.

9. A composition suitable for intravenous administration comprising substantially pure, pyrogen-free D-Ribose.

10. The composition of claim 9 further comprising D-Glucose.

11. The composition of claim 10 comprising 5% to 10% pyrogen-free D-Ribose and 5% to 10% D-Glucose.
